(19) United States
(12) Patent Application Publication
(10) Pub. No.: US 2006/0099586 A1
(43) Pub. Date: May 11, 2006

(54) Eef1a2 for use in the prognosis, diagnosis, and treatment of cancer

(76) Inventor: Jonathan M. Lee, Ottawa Ontario (CA)

Correspondence Address: LICATLA & TYRRELL P.C., 66 E. MAIN STREET, MARLTON, NJ, 08053, US

(21) Appl. No.: 10/516,478

(22) PCT Filed: Jun. 6, 2003
(86) PCT No.: PCT/CA03/00865

§ 371 Date: Jun. 27, 2005

Related U.S. Application Data
(60) Provisional application No. 60/387,231, filed on Jun. 7, 2002.

(51) Int. Cl. C12Q 1/68; G01N 33/574; A61K 48/00; A61K 38/17; A61K 31/7072; A61K 31/704; A61K 31/7048; A61K 31/553; A61K 31/4745; A61K 31/337

(52) U.S. Cl. 435/6; 435/7.23; 514/2; 514/44; 514/34; 514/184; 514/27; 514/49; 514/283; 514/109; 514/449; 514/251; 514/211.08; 424/649

(57) ABSTRACT

Methods and kits for diagnosing and prognosticating cancer via detection of EEF1A2 and/or EEF1A2 are provided. Also provided are methods for treating cancer via inhibition of expression and/or activity of EEF1A2 and screening assays to identify new anticancer agents based upon their ability to inhibit EEF1A2 expression and/or activity. The methods and kits of the present invention are particularly useful in the prognosis, diagnosis and treatment of ovarian, breast and colorectal cancer.

This application claims the benefit of priority from U.S. provisional application Ser. No. 60/387,231, filed Jun. 7, 2002, the teachings of which herein are incorporated by reference in their entirety.

### FIELD OF THE INVENTION

The present invention provides methods and kits for diagnosing and prognosticating cancer via detection of EEF1A2 and/or EEF1A2. The present invention also provides methods for treating cancer via inhibition of expression and/or activity of EEF1A2, screening assays to identify new anticancer agents based upon their ability to inhibit EEF1A2 expression and/or activity, and compositions comprising an inhibitor of EEF1A2 expression and/or activity for use in the treatment of various cancers. The methods, kits and compositions of the present invention are particularly useful in the prognosis, diagnosis and treatment of ovarian and breast cancer, as well as colorectal cancer.

### BACKGROUND OF THE INVENTION

In the year 2001, 25,000 North American women were expected to be diagnosed with ovarian cancer. Over half of the women diagnosed with ovarian cancer are likely to die of this disease.

Amplifications of 20q13 have been identified in both breast and ovarian tumors and have been correlated with poor clinical prognosis and increased tumor aggressiveness in breast and ovarian cancer (Tanner et al. Clin. Cancer Res. 1995 1:1455-1461; Isola et al. Am. J. Pathol. 1995 147:905-911; Courjal et al. Br. J. Cancer 1996 74:1984-1989). The presence of four or more copies of 20q13 has been associated with a decreased five-year survival after diagnosis in women with ovarian cancer (Diebold et al. J. Pathol. 2000 190:564-571). Specifically, a 20-30% fraction of ovarian tumors (Courjal et al. Br. J. Cancer 1996 74:1984-1989; Sonoda et al. Genes Chromosomes Cancer 1997 20:320-328; and Diebold et al. J. Pathol. 2000 190:564-571) and a 20-40% fraction of breast tumors (Kallioniemi et al. Proc. Natl. Acad. Sci. USA 1994 91:2156-2160; Tanner et al. Cancer Res. 1994 54:4257-4260; and Tanner et al. Cancer Res. 1996 56:3441-3445) have an increase in copy number of the 20q13 locus, thus implicating one or more genes at 20q13 in the genesis and progression of ovarian tumors.

Multiple genes map to the 20q13 locus including, but not limited to ZNF217, NABC1 (Collins et al. Proc. Natl. Acad. Sci USA 1998 95:8703-8708), CYP24 (Albertson et al. Nat. Genet. 2000 25:144-146), STK15/BTAK (Bischoff et al. EMBO J. 1998 17:3052-3065) and aurora2 kinase (Bischoff et al. EMBO J. 1998 17:3052-3065). Neither CYP24 nor NABC1 are known to have tumorigenic properties. However, mapping of the breast 20q13 amplicon by CGH (comparative genomic hybridization) suggests that the DNA amplifications center on a 2 Mb region around 20q13.2 and CYP24 (Albertson et al. Nat. Genet. 2000 25:144-146), the gene for vitamin D24 hydrolase (Walters, M. R. Endocri. Rev. 1992 13:719-764), implicating this gene as the so-called “amplicon driver” for 20q13 in breast cancer. In addition, ZNF217 has been disclosed as promoting the immortalization of mammary epithelial cells (Nonet et al. Cancer Res. 2001 61:1250-1254) and STK15 has been disclosed as a transformer (Bischoff et al. EMBO J. 1998 17:3052-3065). Aurora2 is transforming as well and is present in the 20q13 amplicon of colorectal tumors.

Another gene that maps to the 20q13 locus is EEF1A2 (Lund et al. Genomics 1996 36:359-361). EEF1A2 encodes protein elongation factor EEF1A2 (formerly eEF-1α2). During protein translation, eukaryotic elongation factors (EEF) control the recruitment of amino-acylated tRNA to the ribosome and regulate the translocation of the growing polypeptide from the ribosome A to P sites (Hershey et al. Annu. Rev. Biochem. 1991 60:717-755). Human EEF1A2 is one of two isoforms of eukaryotic elongation factor 1 alpha (EEF1A1 and EEF1A2) that share >90% DNA sequence and amino acid identity. EEF1A proteins bind and hydrolyze GTP and catalyze the association of tRNAs to the ribosome during protein elongation (Hershey et al. Annu. Rev. Biochem. 1991 60:717-755). In addition to their role in protein translation, EEF1A proteins from a variety of sources bind to F-actin (Condeelis, J. Trends Biochem. Sci. 1995 20:169-170; Yang et al. Nature 1990 347:494-496) and depolymerize α-tubulin microtubules (Shina et al. Science 1994 266:282-285). Accordingly, it is believed that these proteins have a role in regulating cytoskeletal organization.

A homozygous deletion of the first intron and promoter of the EEF1A2, termed the wst allele, occurs in the Wasted mouse, a spontaneous HRS/J variant (Shultz et al. Nature 1982 297:402-404; Chambers et al. Proc. Natl. Acad. Sci. USA 1998 95:4463-4468). The deletion prevents EEF1A2 transcription (Chambers et al. Proc. Natl. Acad. Sci. USA 1998 95:4463-4468). EEF1A2-deficient Wasted mice suffer a B- and T-cell immuno-deficiency and neuromuscular abnormalities (Shultz et al. Nature 1982 297:402-404) and die by 30 days of age of unknown cause. Wasted mice display an increase in lymphocyte apoptosis relative to EEF1A2+/− animals and the possibility that EEF1A2 may be an inhibitor of apoptosis has been raised (Potter et al. Cell Immunol. 1998 188:111-117).

### SUMMARY OF THE INVENTION

An aspect of the present invention relates to methods for diagnosing and prognosticating various cancers in a subject comprising measuring EEF1A2 or EEF1A2 levels in a biological sample obtained from the subject and comparing the measured EEF1A2 or EEF1A2 levels with levels of EEF1A2 or EEF1A2 in a control wherein an increase in the measured EEF1A2 or EEF1A2 levels as compared to the control is indicative of the subject having cancer.

Another aspect of the present invention relates to kits for detecting EEF1A2 or EEF1A2 levels in a biological sample for use in diagnosing and prognosticating cancer in a subject.

Another aspect of the present invention relates to antisense oligonucleotides and methods of using the antisense oligonucleotides to inhibit EEF1A2 in a tumor cell.

Another aspect of the present invention relates to methods for treating various cancers comprising administering to a patient suffering from cancer an inhibitor of EEF1A2 expression and/or activity.

Yet another aspect of the present invention relates to screening assays to identify new anticancer agents based upon the ability of an agent to inhibit EEF1A2 expression and/or activity.

### DETAILED DESCRIPTION OF THE INVENTION

The genetic amplification of growth enhancing genes plays a key role in the development of human malignancy. Important to the understanding of oncogenesis is the identification of genes whose copy number and expression increases during tumorigenesis. Agents that functionally inactivate these genes or proteins encoded thereby can be used as anticancer therapeutics. Furthermore, the genes and their RNA and protein products can be used as diagnostic and prognostic markers for disease progression and outcome prediction.

As demonstrated herein, the present inventors have now found that EEF1A2, the gene encoding protein elongation factor EEF1A2 (eEF-1α2), is amplified in various tumors. Further, as also demonstrated herein, the present inventors have also now found that EEF1A2 has properties of an oncogene in that it enhances focus formation, allows anchorage independent growth and decreases the doubling time of fibroblasts, promotes in vivo tumorigenicity in fibroblasts and increases the growth rate and in vivo tumorigenicity of ovarian carcinoma cells when xenografted into nude mice.

In particular, as shown herein by the inventors, EEF1A2, the gene encoding EEF1A2 (formerly eEF-1α2), is genetically amplified in 26% of primary ovarian and 25% of breast and colorectal tumors. In addition, as shown herein EEF1A2 amplification correlates with significantly reduced survival among ovarian cancer and breast cancer patients. EEF1A2 mRNA levels are also increased in 27% of primary ovarian tumors and 33% of established cell lines. The strong transforming and tumorigenic properties of EEF1A2 are indicative of this gene and the protein encoded thereby having an important role in oncogenesis over and above any potential role as a 20q13 amplicon driver.

Further, the inventors have now found that EEF1A2 has growth-promoting properties. Expression of EEF1A2 alters the growth properties of mouse NIH 3T3 fibroblasts by increasing their growth rate and allowing them to grow in an anchorage-independent manner in soft agar. Expression of EEF1A2 in RAT1 fibroblasts causes these cells to grow as a multi-layered focus. Anchorage-independent growth and focus formation are characteristics of cancerous cells. Importantly, expression of EEF1A2 in NIH 3T3 cells makes these cells tumorigenic in mice. Expression of EEF1A2 in the human ES2 ovarian carcinoma line increases the ability of these human cells to grow as tumors in nude mice. Thus, it is believed that EEF1A2 is an oncogene, a gene that promotes cancer development.

To determine whether EEF1A2 is part of the 20q13 amplicon in ovarian cancer, FISH (fluorescence in situ hybridization) was used to measure EEF1A2 copy number in primary ovarian tumors. It was found that a 25% subset of primary ovarian tumors (14/53) have EEF1A2 gene amplifications. Amplifications of EEF1A2 were visualized by the increased number of loci hybridizing to an EEF1A2 BAC (bacterial artificial chromosome) probe. The BAC probe contains the EEF1A2 3′ UTR as determined by PCR. Hybridization of a control 20p11 probe to the same samples indicates that the increase in EEF1A2-hybridizing loci does not result from chromosome 20 polyploidy. By chromosomal metaphase spread, it was shown that the BAC clone used for the FISH hybridizes to 20q13. Thus, EEF1A2 copy number is increased in a substantial subset of ovarian tumors and is part of the 20q13 amplicon.

FISH (fluorescence-in situ hybridization) was also used to demonstrate that approximately 25% of primary human breast tumors (6 of 26) exhibit EEF1A2 gene amplification and approximately 29% of colorectal cancers (4 of 14) exhibit EEF1A2 gene amplification. In contrast, EEF1A2 gene amplification was not observed in any prostate tumors (0 of 17).

To determine whether there is an increase in EEF1A2 expression in ovarian tumors, northern blotting was used to measure EEF1A2 mRNA levels in primary ovarian tumors and established ovarian carcinoma cell lines. Although the tissue-specific expression pattern of human EEF1A2 is currently unknown, rat and mouse EEF1A2 RNA is expressed only in normal brain, heart and skeletal muscle (Lee et al. J. Biol. Chem. 1992 267:24064-24068; Knudsen et al. Eur. J. Biochem. 1993 215:549-554). EEF1A2 message was undetectable in normal ovarian tissue, whereas 3/11 primary ovarian tumors had readily detectable EEF1A2 RNA. GAPDH and EEF1A1 gene expression was similar among the samples. The EEF1A1 gene (EEF1A1) maps to 6q14, a locus not known to be involved in ovarian cancer. Further, 2 out of 3 tumor samples with elevated EEF1A2 mRNA had increased EEF1A2 copy number. One of the tumor samples with elevated EEF1A2 expression did not have detectable EEF1A2 amplification, suggesting that EEF1A2 expression may increase independently of EEF1A2 copy number changes. EEF1A2 mRNA expression is also increased in some established ovarian cancer cell lines relative to normal ovarian epithelial cells. The normal ovarian epithelial cell line, NOV-61, had undetectable EEF1A2 RNA. In contrast, 4 out of 12 ovarian tumor cell lines (TOV112D, PA1, HEY, 2008) expressed EEF1A2. The OV-90, TOV81D, TOV21G, OVCAR3, OVCAR4, CAOV3, SKOV3 and ES-2 cells lines, like the normal NOV-61 cell line, did not detectably express EEF1A2 mRNA. GAPDH and EEF1A1 gene expression was similar among the cell lines. Taken together, these data indicate that EEF1A2 expression is increased in an approximate 30% subset of ovarian tumor samples and cell lines.

Semi-quantitative RT-PCR (reverse-transcriptase-polymerase chain reaction) was used to estimate EEF1A2 RNA expression in primary breast tumors. EEF1A2 RNA was undetectable in normal breast tissue, but 2 out of 6 primary human breast tumors had readily detectable EEF1A2 RNA expression. Actin expression was similar among all samples. The increased EEF1A2 copy number and RNA expression in primary tumors implicates EEF1A2 in breast cancer development.

The oncogenic properties of human EEF1A2 were also assessed. For these experiments, NIH 3T3 rodent fibroblast cell lines were established by stably expressing EEF1A2 under the control of the CMV promoter. The EEF1A2 used to generate the cell lines was tagged at its carboxy-terminus with the V5 epitope (Gly-Lys-Pro-Ile-Pro-Asn-Pro-Leu-Leu-Gly-Leu-Asp-Ser-Thr (SEQ ID NO:1)) to facilitate detection by western blotting. Protein expression of exogenous EEF1A2 was determined in three independent NIH 3T3 clones (N-1,N-2,N-3). The EEF1A2 protein in interphase cells is non-nuclear and diffusely cytoplasmic, corresponding to the wild type localization of the protein (Kjaer et al. Eur. J. Biochem. 2001 268:3407-3415). The EEF1A2-expressing clones grew as colonies in soft agar, a property not observed in the parental NIH 3T3 cells or NIH 3T3 cells transfected with the empty vector. Moreover, the EEF1A2-expressing clones had an accelerated growth rate relative to the parental NIH 3T3 controls. Four days after plating an equal number of cells, there were approximately four times as many EEF1A2-expressing cells as parental cells, indicating that EEF1A2 expression enhances cell growth rate.

The capacity of EEF1A2 to enhance cell growth was also assessed by measuring the ability of EEF1A2 to induce focus formation in Rat1 fibroblasts. The ability to form foci in cell culture is a marker for cell transformation and is considered one of the general properties of an oncogene such as RAS (Land et al. Nature 1983 Nature 304:596-602). EEF1A2 induced focus formation in Rat1 cells. The constitutively active and transforming RAS^{va112} allele (Provencher et al. In Vitro Cell Dev. Biol. Anim. 2000 36:357-361) was used as a positive control. The morphology of EEF1A2-induced foci was similar to those induced by RAS^{val12}.

To determine whether EEF1A2 enhanced tumorigenicity, EEF1A2-expressing NIH 3T3 cells were subcutaneously injected into nude mice. Expression of EEF1A2 in NIH 3T3 cells was sufficient to induce in vivo tumorigenicity. No tumor growth was observed in the parental or vector-transfected NIH 3T3 cells. While the N-1 line expressed more EEF1A2 protein than either N-2 or N-3, it did not appear to form larger tumors in the mice nor was it more efficient at forming colonies in soft agar. This indicates that N-1, N-2 and N-3 are expressing enough EEF1A2 protein so that its abundance is not the limiting factor in either anchorage-independent growth or in vivo tumorigenesis.

To determine the effect of EEF1A2 on an ovarian-derived cell, independent ES-2 ovarian cell lines that express EEF1A2 (E-1,E-2,E-3,E-4) were generated. ES-2 are ovarian clear cell carcinoma cells that do not express detectable EEF1A2 mRNA. Protein expression of EEF1A2 was determined in four independent ES-2 derivatives. A non-specific background band of slightly higher molecular weight than the EEF1A2 protein was seen in the parental and vector lanes and could also be discerned in the E-1, E-2, and E-3 lysates. The cell lines expressing EEF1A2 all had an accelerated rate of tumor formation in nude mice relative to the ES-2 controls. Thus, EEF1A2 enhanced their in vivo tumorigenicity. Representative sections of ES-2-derived tumors were stained with hematoxylin and eosin. All tumors showed high-grade malignancy with an ischemic necrotic core indicative of rapid tumorigenesis.

The effects of EEF1A2 on the growth and tumorigenicity of human breast cancer cells lines can also be examined. For these experiments, human breast cancer cell lines that over-express human EEF1A2 are developed. Since a fraction of primary breast tumors have high levels of EEF1A2 expression, three breast cancer cell lines that do not express endogenous EEF1PA2 mRNA must first be identified. To this end, northern blotting is preferably used to measure EEF1A2 RNA expression in human breast cancer cell lines such as MCF-7, MDA, Hs-274, Hs-280, Hs-343, Hs-362, Hs-386, Hs-739, Hs-741Hs-743, Hs-823, Hs-902, MB-157, UACC-12, HCC1008, HCC1954, BT-483, T-47D, Hs-54, HCC2157 and HCC1937 relative to expression in the normal breast epithelial cell lines CCD-986 and CCD1056. These normal and malignant lines represent a spectrum of breast tissue types and are all available from the ATCC. However, as will be understood by those of skill in the art upon reading this disclosure, alternative breast cancer cell lines can be used as well. The three different EEF1A2-non-expressing tumor cell lines are then transfected with a human EEF1A2 gene that is under the transcription control of the CMV promoter. This is the same plasmid construct used to demonstrate that EEF1A2 expression increases the tumorigenicity and in vitro growth of mouse 3T3 fibroblasts. At least three independent EEF1A2-expressing clones are derived for each cell type. Expression of the exogenous EEF1A2 after selection is determined preferably by western blotting. EEF1A2-expressing tumor cell lines are then tested for their ability to grow in soft agar. Their doubling time in 10% serum and reduced serum and plating efficiency is measured as well. To determine whether EEF1A2 can directly enhance tumorigenesis EEF1A2-expressing breast cancer cell lines are injected subcutaneously into nude mice and tumor volume relative to parental non-EEF1A2-expressing cells is measured as a function of time following injection (Ozzello, L. Prog. Clin. Biol. Res. 1977 12:55-70).

The effect of EEF1A2 on the growth and tumorigenicity of normal breast epithelial cell lines can also be examined. For these experiments, normal breast epithelial cell lines expressing EEF1A2 are derived. Since normal epithelial cells are generally refractory to plasmid transfection, an adenovirus EEF1A2 vector is first derived in accordance with methods such as described by He et al. (Proc. Natl. Acad. Sci. 1998 95:2509-2514). The EEF1A2 adenovirus is then used to infect a breast epithelial line that does not normally express EEF1A2, as identified above. EEF1A2 cell lines are assayed for in vitro growth and tumorigenicity as described above for the malignant cells. Since normal breast epithelial cells generally have a finite life-span, the doubling potential of an EEF1A2 expressing cell line can also be compared relative to that of a cell line infected with a control lacz adenovirus.

The demonstrated ability herein of EEF1A2 promoting cancerous growth is indicative of EEF1A2 being a target for anti-cancer therapy. It is believed that EEF1A2 inactivation through inhibition of expression of EEF1A2 and/or through inhibition of the activity of this protein will slow or stop the growth of cancer cells. Accordingly, one aspect of the present invention relates to methods of treating cancer by administering an agent that inhibits EEF1A2 expression and/or activity. Such EEF1A2 inactivating agents are expected to be particularly useful in the treatment of ovarian cancer. Other cancers, including breast and colorectal cancer, are also expected to be targets for EEF1A2 inactivation.

As used herein, “expression of EEF1A2”, i.e., gene expression, refers to either or both mRNA products of the EEF1A2 gene and consequent protein products. Gene expression may be measured therefore by measuring mRNA and/or protein levels. Alternatively, expression may be monitored by assaying protein activity.

In one embodiment of a method for treatment, an anticancer agent comprises an antisense oligonucleotide, which hybridizes to EEF1A2 or mRNA thereof and inhibits transcription of EEF1A2 and/or protein translation of EEF1A2 mRNA. An antisense oligonucleotide can comprise a nucleotide sequence which is complementary to a coding strand of a nucleic acid, e.g. complementary to an mRNA sequence, constructed according to the rules of Watson and Crick base pairing, and can hydrogen bond to the coding strand of the nucleic acid. The antisense sequence complementary to a sequence of an mRNA can be complementary to a sequence found in the coding region of the mRNA or can be complementary to a 5′ or 3′ untranslated region of the mRNA. Furthermore, an antisense oligonucleotide can be complementary in sequence to a regulatory region of the gene encoding the mRNA, for instance a transcription initiation sequence or regulatory element. Preferably, an antisense nucleic acid complementary to a region preceding or spanning the initiation codon or in the 3′ untranslated region of an mRNA is used. An antisense nucleic acid can be designed based upon the nucleotide sequence shown in SEQ ID NO: 5. A nucleic acid is designed which has a sequence complementary to a sequence of the coding or untranslated region of the shown nucleic acid.

The antisense oligonucleotides of the invention can be constructed using chemical synthesis and enzymatic ligation reactions using procedures known in the art. The antisense oligonucleotide can be chemically synthesized using naturally occurring nucleotides or variously modified nucleotides designed to increase the biological stability of the molecules or to increase the physical stability of the duplex formed between the antisense and sense nucleic acids e.g. phosphorothioate derivatives, acridine substituted nucleotides, and 2′-O-propyl modified nucleotides can be used. Alternatively, the antisense oligonucleotides can be produced biologically using an expression vector into which a nucleic acid has been subcloned in an antisense orientation (i.e. nucleic acid transcribed from the inserted nucleic acid will be of an antisense orientation to a target nucleic acid of interest). The antisense expression vector is introduced into cells in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

In some embodiments of the present invention, the antisense oligonucleotide may be formulated in liposomes and delivered to target cells. Methods of manufacturing liposomes are described, e.g., in U.S. Pat. No. 4,522,811, No. 5,374,548 and No. 5,399,331. The liposomes may comprise one or more targeting moieties that are selectively transported into specific cells or organs (see, e.g., Ranade, J. Clin. Pharmacol. 1989 29: 685). Exemplary targeting moieties include folate, biotin, mannosides, antibodies, surfactant protein A receptor, and gp120. The antisense oligonucleotides may be subjected to electrostatic liposome encapsulation (NeoPharm Inc., Lake Forest, Ill.; Gokhale et al. Clinical Cancer Research 2002 8:3611-3621). Such liposomes may comprise a targeting moiety, e.g., a tumor-specific monoclonal antibody.

The antisense oligonucleotides of the present invention are useful for inhibiting expression of nucleic acids (e.g. mRNAs) encoding proteins having EEF1A2 activity, thereby decreasing expression of proteins having EEF1A2 activity. Since increased expression of proteins having EEF1A2 activity is associated with and can confer oncogenic properties on a cell including enhancing focus formation, allowing anchorage independent growth and decreasing the doubling time of fibroblasts, promoting in vivo tumorigenicity in fibroblasts and increasing the growth rate and in vivo tumorigenicity of carcinoma cells xenografted into nude mice, decreasing expression of such proteins can inhibit or reverse such oncogenic properties of the cell into which the antisense oligonucleotide has been introduced. Antisense oligonucleotides can be introduced in to a cancer cell, preferably an ovarian, breast or colorectal cancer cell in culture to inhibit EEF1A2 expression. One or more antisense nucleic acids, such as oligonucleotides, can be added to cells in culture media, typically at 10 to 1000 μg/ml. A cultured cancer cell in which EEF1A2 expression is inhibited is useful for testing the efficacy of potential therapeutic agents. For example, EEF1A2 expression could be inhibited in a tumor cell line that expresses EEF1A2 to determine the contribution of EEF1A2 to an observed response of the cell to a particular therapeutic agent.

The antisense oligonucleotides of the present invention can also be used in gene therapy to correct or prevent EEF1A2 expression in a subject. For example, antisense sequences can be used to render malignant cells incapable of expressing EEF1A2. Correction or prevention of EEF1A2 expression is expected to be useful in the treatment of cancers, and in particular ovarian, breast and colorectal cancers. Administration of antisense nucleic acids to a subject may be most effective when the antisense oligonucleotide is contained in a recombinant expression vector which allows for continuous production of antisense RNA. Recombinant molecules comprising an antisense oligonucleotide, can be directly introduced into tissues, including ovarian, breast or colorectal tissue in vivo, using delivery vehicles such as liposomes, retroviral vectors, adenoviral vectors and DNA virus vectors. A delivery vehicle can be chosen which can be targeted to a cell of interest in the subject (e.g. a ovarian, breast or colorectal tumor cell). Antisense oligonucleotides can also be introduced into isolated cells, such as those of the hematopoietic system, ex vivo using viral vectors or techniques such as microinjection, electroporation, coprecipitation and incorporation of DNA into liposomes (e.g. lipofectin) and such cells can be returned to the donor. Recombinant molecules can also be delivered in the form of an aerosol or by lavage. Antisense oligonucleotides can also be delivered by other routes including, but not limited to intravenously, orally, topically, rectally, intramuscularly and intraperitoneally, in accordance with well known procedures.

Antisense inactivation of EEF1A2 was achieved using two EEF1A2-specific phosphorothioated antisense oligonucleotides. Phosphorothioated DNA oligonucleotides were used as these modified oligonucleotides are non-toxic in humans, nuclease resistant, stable in vivo and in vitro, are readily taken up by cells and activate rapid degradation of the mRNA target (Gewirtz et al. Blood 1998 92:712-736; Agrawal et al. Proc. Natl. Acad. Sci. USA 1990 87:1401-1405). However, as will be understood by those of skill in the art upon reading this disclosure, other modifications well known to those skilled in the art can be used.

These antisense oligonucleotides comprised the following sequences:

| | |
|---|---|
| CTTTTGCTGGGAGTGTGAGG; | (SEQ ID NO:2) |
| and | |
| GCTGGGAGTGTGTGAGGGGCTG. | (SEQ ID NO:3) |

Both antisense oligonucleotides decreased EEF1A2 mRNA levels in human Ramos cells. mRNA levels of EEF1A1 were not decreased by administration of these antisense oligonucleotides. A control sequence, GGTTGCTGTGGGCTTGAGT (SEQ ID NO:4) had no effect on mRNA levels of EEF1A2 or EEF1A1 in the human Ramos cells.

Accordingly, the invention provides a method for inhibiting EEF1A2 of a tumor cell, preferably an ovarian, breast or colorectal tumor cell by introducing into the tumor cell a nucleic acid which is antisense to a nucleic acid which encodes the protein shown in SEQ ID NO: 6. In a preferred embodiment, the antisense oligonucleotide comprises SEQ ID NO:2 or SEQ ID NO:3. Further, the method may comprise administration of a second therapeutic drug, preferably a second anticancer drug such as a taxane, which as taught infra, is expected to have reduced efficacy in tumor cells expressing EEF1A2. Accordingly, administration of an antisense oligonucleotide that inhibits EEF1A2 in combination with an anticancer drug, preferably a taxane, is expected to enhance efficacy of the anticancer drug.

Additional antisense oligonucleotides to those exemplified herein, also capable of hybridizing to EEF1A2 or mRNA thereof and inhibiting transcription of EEF1A2 and/or protein translation of EEF1A2 mRNA can be identified routinely by those skilled in the art in accordance with the teachings herein.

The specific inactivation of EEF1A2 requires an antisense molecule that binds to EEF1A2 (Genbank Accession No. NM_{—}001958; SEQ ID NO:5) but not the closely related EEF1A1 (Genbank Accession No. NM_{—}001402; SEQ ID NO:7). The mRNA coding sequence of EEF1A1 and EEF1A2 are >90% identical. The 5′ and 3′ ends of the human EEF1A2 mRNA are the most different from EEF1A1 mRNA, making the mRNA termini preferred targets for specific inactivation of EEF1A2 and design of additional antisense oligonucleotide. To identify additional useful EEF1A2 specific antisense oligonucleotide, approximately 15 EEF1A2 specific antisense molecules are prepared; 9 complementary to the 5′ end (residues 1-39 and 52-78) and 6 complementary to the 3′ end (residues 1467-1500).

Northern blotting is used to measure the ability of each antisense, in doses of 1-20 μM, to decrease EEF1A2 but not EEF1A1 mRNA. GenePorter (Gene Therapy Systems) is preferably used to deliver the antisense oligonucleotide since its delivery efficiency is >80% with minimal cytotoxicity. Transfection reagents are not required for in vivo delivery. Three ovarian or breast cancer cell lines that have high EEF1A2 expression are used as well as one non-EEF1A2-expressing cell line to determine the ability of each antisense to reduce EEF1A2 but not EEF1A1 mRNA, to halt cell cycle (propidium iodide staining), induce apoptosis (Annexin V staining; vanEngeland et al. Cytometry 1998 31:1-9) and decrease overall viability (trypan blue exclusion) of the cancer cell lines.

Because germline EEF1A2 inactivation causes immune dysfunction and mortality in Wasted mice (Shultz et al. Nature 1982 297:402-404), the antisense oligonucleotides are tested for systemic toxicity in nude and normal C57BL/6 mice. The goal is to determine the maximum tolerated dose of each antisense compound. To this end, mice (n=4/dose) are treated with decreasing doses, in saline, from 6.0 mg/kg i.v. every day for 2 weeks. This dose regimen represents the maximum tested dose for inhibition of tumorigenesis by a c-raf antisense in a mouse model of A549 lung carcinoma model (Monia et al. Nat. Med. 1996 2:668-675) and is used as an experimental starting point. Dose can be increased if the antisense is well tolerated. During treatment the mice are observed daily for malaise, mobility difficulties or other treatment side effects. The amount of circulating antibodies and peripheral B- and T-cells are monitored weekly using ELISA assays. Animals are also weighed daily. At the conclusion of treatment, organs are weighed. Antisense oligonucleotides that do not show evidence for toxicity are then further studied.

For example, the anti-breast or anti-ovarian tumor activity of the antisense oligonucleotide is examined in mice injected subcutaneously with two independent EEF1A2-expressing human breast or ovarian cancer cells. After a measurable tumor volume (>5 mm in diameter) has appeared, the antisense molecule is delivered intravenously to the reconstituted mice at the highest non-toxic dose as determined above (n=6 mice/dose). Tumor volume is monitored daily. As appropriate, a minimum effective dose is determined. The ability of the antisense oligonucleotide to halt or reverse tumor progression is indicative of its efficacy.

The pharmacokinetic properties of the antisense in mice can also be examined using ³²P-labeled antisense delivered iv. Plasma half-life and urine and fecal clearance rates can be determined as well as accumulation rates in the brain, thymus, spleen, bone, liver, and kidney.

The nucleic acids of the invention can further be used to design ribozymes which are capable of cleaving a single-stranded nucleic acid encoding a protein having EEF1A2 activity, such as an mRNA. A catalytic RNA (ribozyme) having ribonuclease activity can be designed which has specificity for a EEF1A2-encoding mRNA based upon the sequence of a nucleic acid of the invention. For example, a derivative of a Tetrahymena L-19 IVS RNA can be constructed in which the base sequence of the active site is complementary to the base sequence to be cleaved in a EEF1A2-encoding mRNA. See for example Cech et al. U.S. Pat. No. 4,987,071: Cech et al. U.S. Pat. No. 5,116,742. Alternatively, a nucleic acid of the invention could be used to select a catalytic RNA having a specific ribonuclease activity from a pool of RNA molecules. See for example Bartel, D. and Szostak, J. W. Science 261, 1411-1418 (1993).

Short interfering RNA (siRNA) can be also be used to inhibit EEF1A2. siRNA is a relatively new technology that silences gene expression through a process referred to as RNA interference (RNAi) (Moss, E. G. Curr. Biol. 2001 11:R772-R775; Carthew, R. W. Curr. Opin. Cell Biol. 2001 13:244-248). RNAi depends on the formation of double stranded RNA (dsRNA) derived from coding sequences of the gene to be silenced (Moss, E. G. Curr. Biol. 2001 11:R772-R775; Carthew, R. W. Curr. Opin. Cell Biol. 2001 13:244-248). Gene expression is halted through a combination of target gene methylation (Wassenegger et al. Cell 1994 76:567-576) and post-transcriptional degradation of the gene's mRNA (Hammond et al. Nature 2000 404:293-296). RNAi can be experimentally activated through the use of siRNA, duplexes of 21-25 base pair RNA fragments that are complementary to the gene being silenced (Elbashir et al. Nature 2001 411:494-498). siRNA can be used to inactivate gene expression in cultured mammalian cell lines (Caplen et al. Proc. Natl. Acad. Sci. USA 2001 98:9742-9747; Cogoni, C. and Macino, G. Curr. Opin. Genet. Dev. 2000 10:638-643) and in multiple organs of postnatal mice (Lewis et al. Nat. Genet. 2002 32:107-108). siRNA-mediated gene-silencing effect is usually achieved using double-stranded siRNA 21 nucleotides in length. siRNA usually consist of a 19-nucleotide complementary region and a two nucleotide non-complementary 3′ overhang. An effective RNAi target sequence is likely to be 50-100 nucleotides downstream of the start codon. siRNA are commercially synthesized (Xeragon) and then transfected into EEF1A2-expressing breast or ovarian tumors cells using TransMessenger (Qiagen), a lipid based transfection agent designed specifically for delivery of RNA. Northern and western blots are used to determine EEF1A1 and EEF1A2 mRNA and protein levels. A plasmid based vector system is used to generate siRNA since a plasmid vector is generally deliverable to animals either on its own or incorporated into a virus. Like the antisense experiments described above, the ability of each siRNA to reduce EEF1A2 mRNA, to halt cell cycle, induce apoptosis and decrease overall viability of the breast or ovarian cancer cell lines is determined.

Other anticancer agents useful in the present invention may comprise small organic molecules, proteins, peptides or peptidomimetics that are capable of inactivating or inhibiting EEF1A2. High-throughput technology is used to screen libraries of several thousand compounds, e.g. natural product libraries, to identify inactivators or inhibitors of EEF1A2. The screen assays the effect that each compound of the library has on the growth rate of cultured cell lines, preferably EEF1A2-overexpressing cells. Screening with high-throughput technology is currently ongoing and EEF1A2 inhibition has been detected in a library of compounds. Like the antisense experiments described above, the ability of each small molecule to halt cell cycle, induce apoptosis and decrease overall viability of the breast or ovarian cancer cell lines is then determined. In addition, the ability of the molecule to inhibit enhancement of the translation of polyPhe mRNA by purified EEF1A2 in rabbit reticulocyte lysates, to bind and hydrolyze radiolabelled GTP or to co-precipitate with F-actin, is assessed. Such in vitro assays are all well-characterized for assessing EEF1A function.

The present invention also relates to screening assays for use in identifying potential anticancer agents based upon their ability to inactivate or inhibit EEF1A2. For example, a screening assay of the present invention may comprise individually testing potential anticancer agents for their ability to inhibit: a) EEF1A2-mediated enhancement of NIH 3T3 cell growth; b) EEF1A2-mediated enhancement of protein translation; and/or c) EEF1A2-mediated microtubule cleavage. The ability of a test agent to inhibit one or more of these activities is indicative of the agent being useful in the treatment of cancer, particularly ovarian, breast or colorectal cancer.

Another aspect of the present invention relates to the use of EEF1A2 gene amplification, EEF1A2 mRNA levels or EEF1A2 protein levels or activity as a prognostic marker in cancer, particularly ovarian cancer, as well as breast and colorectal. Ovarian cancer patients with EEF1A2 amplification survived a shorter period of time following diagnosis than ovarian cancer patients without EE1FA2 amplification. EEF1A2 gene amplification is also associated with decreased probability of 10-year survival in breast cancer patients. Thus, detection of EE1FA2 amplification or increases in EE1FA2 mRNA levels or EEF1A2 proteins levels or activity can be used to prognosticate survival time of a cancer patient.

FISH and immunohistochemistry can be used as well to determine the extent to which EEF1A2 gene amplification and protein expression correlate with 11-year relapse-free survival, 11-year survival and breast tumor size, grade, nodal status, grade, estrogen receptor (ER) status, Her2 status, and lymphovascular invasion in a cohort of >1500 Canadian breast tumor samples (Parker et al. Am. J. Clin. Pathol. 2002 117:723-728). More specifically, EEF1A2 can be linked to the above listed clinical and pathological parameters using a tissue microarray (TMA) of >5000 Canadian breast tumor samples. TMA are composed of 600 μm diameter cylindrical samples taken from different archival tissue blocks and placed into a single empty recipient paraffin block (Bubendorf et al. J. Pathol. 2001 195:72-79; Kononen et al. Nat. Med. 1998 4:844-847). A TMA typically contains several hundred different tumor samples that can be simultaneously analyzed by immunohistochemistry or in situ hybridization in a single TMA section on a single standard microscope slide. TMA technology has proved effective at analyzing the molecular pathology of breast, bladder, and prostate cancer and substantially reduces the time required to establish correlations between tumor pathology and molecular biology. For this analysis, a TMA of >5000 Canadian breast tumor samples will be used which have 11-year relapse-free survival, 11-year survival and breast tumor size, grade, nodal status, grade, estrogen receptor (ER) status, Her2 status, and lymphovascular invasion data associated with them (Parker et al. Am. J. Clin. Pathol. 2002 117:723-728).

In one embodiment, the presence of EEF1A2 amplifications in primary ovarian and breast tumors is identified using an EEF1A2-containing bacterial artificial chromosome (BAC). The presence of EEF1A2 amplifications can be used as a genetic marker to predict the probability of survival.

In another embodiment EEF1A2 protein expression can serve as the prognostic marker of ovarian, breast or colorectal cancer. For example, the EEF1A2 protein is not expressed in normal ovarian epithelial cells or breast tissue. Thus, antibodies that specifically recognize EEF1A2 protein can be generated and used to stain samples of tumor removed from ovarian cancer or breast cancer patients.

In one embodiment, to produce these polyclonal antibodies, rabbits are immunized with the SHTTLLEAVDCIL (SEQ ID NO:8) peptide conjugated to KLH (keyhole limpet hemocyanin). This peptide, which corresponds to EEF1A2 residues 224-236, contains 4 amino acid differences between EEF1A1 and EEF1A2, and is found in a predicted hinge region between the actin binding and tRNA binding domains. EEF1A1 specific antibodies from the immunization are absorbed using an Affigel affinity column containing SGVSLLEALDTIL (SEQ ID NO:9; differences underlined), the EEF1A1 peptide. Specificity of the antibody is confirmed by immunoblotting with a GST-EEF1A1 and a GST-EEF1A2.

Patients with tumor samples that are positive for EEF1A2 are expected to survive for a shorter period of time as compared to patients with tumor sample negative for EEF1A2. Prognostic information relating to EEF1A2-gene amplification and/or EEF1A2 protein expression can be used to enhance clinical decision-making and to select appropriate treatment regimes. NIH 3T3 cells ectopically expressing EEF1A2 are resistant to the apoptosis-induced by cisplatin and staurosporine. Further, mice lacking EEF1A2 show increased lymphoid apoptosis. Accordingly, it is believed that EEF1A2 modulates the sensitivity of cancer cells to selected treatments.

Furthermore, since EEF1A can directly or indirectly cause microtubule severing (Shiina et al. Science 1994 266:282-285), it is believed that EEF1A2 modulates the cytotoxicity of taxane compounds since their cytotoxicity stems from their ability to stabilize microtubules. In particular, EEF1A2 expression is expected to increase resistance to taxol-induced cell death and microtubule stabilization and thus is prognostic of a poor response to taxol. Examples of other anticancer treatments, efficacy of which may be modulated by the EEF1A2 expression include, but are not limited to, classes of compounds such as anthracyclines, epipodophyllotoxins, vinca alkaloids, metallocenes (such as platinum-based compounds), of which cyclophosphamide, methotrexate, fluorouracil, doxorubicin, epirubicin, paclitaxel and cisplatin are examples.

In addition, increased EEF1A2 expression in tumors such as primary ovarian tumors in a subject is expected to lead to increased EEF1A2 protein levels in biological samples such as blood and other tissues obtained from the subject. Accordingly, measurement of increased EEF1A2 levels in a biological sample such as plasma, serum or other tissue obtained from a subject can be used as a diagnostic tool for cancers such as ovarian, breast and colorectal cancer in the subject. Blood or other tissue samples can be taken from a subject and analyzed for the presence of EEF1A2 protein using a standard immunoassay technique such as an ELISA with an EEF1A2-specific antibody. Measured levels of EEF1A2 protein in the sample can then be compared to levels in a control. As used herein, by “control” it is meant, a sample obtained from an individual known not have cancer, a sample obtained previously from the subject prior to the onset or suspicion of cancer, or a standard from data obtained from a data bank corresponding to currently accepted normal levels of this gene or gene product. Increased EEF1A2 protein levels in the sample obtained from the subject as compared to levels in the control are indicative of the subject having ovarian, breast or colorectal cancer. The comparison performed may be a straight-forward comparison, such as a ratio, or it may involve weighting of one or more of the measures relative to, for example, their importance to the particular situation under consideration. The comparison may also involve subjecting the measurement data to any appropriate statistical analysis.

Another aspect of the present invention relates to kits for diagnosing and prognosticating cancer in a subject by detecting EEF1A2 gene amplification or EEF1A2 expression and/or activity. Kits for detection of EEF1A2 gene amplification preferably comprise a means for detection such as a EEF1A2-containing bacterial artificial chromosome (BAC) as well as instructions for use of BAC in detecting EEF1A2 gene amplification in tumor tissue samples. Kits for detection of EEF1A2 mRNA levels preferably comprise a means for detection such as, for example, northern blotting or a gene chip, as well as instruction for use of northern blotting or the gene chip in detecting EEF1A2 mRNA levels. Kits for detection of EEF1A2 protein levels preferably comprise a means for detection such as an antibody specific for EEF1A2 as well as instructions for use of such antibody to immunoassay a biological sample such as a tumor tissue biopsy sample, or a serum or blood sample obtained from a subject for the presence of EEF1A2. Other components included in these kits may comprise EEF1A2 standards, diluting solutions, and/or wash buffers routinely provided in diagnostic and prognostic kits of this nature.

The following nonlimiting examples are provided to further illustrate the present invention.

### EXAMPLES

### Example 1

### Fluorescence Hybridization and Microscopy

Fluorescence in situ hybridization (FISH) was performed in accordance with the procedure described by Demetrick, D. J. (Mod. Pathol. 1996 9:133-136). In these experiments, EEF1A2 and 20p11 BAC clones were labeled with FITC-dUTP and Digoxygenin (DIG)-dUTP, respectively. The labeled clones were then hybridized at 37° C. to interphase nuclei from frozen ovarian carcinoma tissue samples. Slides were counter-stained with DAPI and visualized utilizing a Zeiss Axioplan 2 microscope. A Photometrics PXL 1400 CCD camera was used to capture images of representative interphase nuclei and Electronic Photography version 1.3 Biological Detection software used for alignment. Adobe PhotoShop was used to pseudocolor FITC and DIG labeled probes. A V5 antibody (InVitrogen) diluted 1:500 in phosphate buffered saline (PBS) followed by an Alexa 546-conjugated (1:200 in PBS) secondary antibody was used to determine EEF1A2 localization.

### Example 2

### RNA Purification and Northern Blotting

Ovarian tumor samples were obtained from the Gynecology and Oncology Group of the Cooperative Human Tissue Network. RNA was prepared from 100-200 mg of frozen tumor homogenized in 2 ml of TriZol (Gibco) as per the manufacturer's directions. RNA from cell lines was obtained through lysis of a 60 mm plate with 1 ml of Trizol (Gibco). 10 ug of total RNA was loaded per lane and RNA was transferred to GeneScreen. Normal ovary mRNA was obtained from Stratagene. Membranes were pre-hybridized at 63° C. in 25 ml Church's Buffer, hybridized in 15 ml Church's at 59° C. overnight, and washed at 62° C. The EEF1A2 probe was a 598 BamHI/PstI fragment of the human EEF1A2 cDNA.

### Example 3

### Cell Culture and Western Blotting

NIH 3T3 and ES-2 cells were grown in 10% FBS/DMEM and 10% FBS/McCoy's 5A respectively. EEF1A2-expressing NIH 3T3 and ES-2 cells were derived by transfecting NIH 3T3 cells with 5 ug of the EEF1A2 plasmid and 15 ul of SuperFect (Qiagen) per 60 mm dish. 0.4 mg/ml Zeocin (InVitrogen) was used to select transfectants and independent clones derived by limiting dilution cloning. An α-V5 antibody (InVitrogen; 1:500 in TBST) followed by an HRP conjugated goat anti-mouse (BioRad; 1:1,000 in TBST) and ECL+ (Amersham) were used to detect EEF1A2 expression. Cell growth was measured by Coulter counting triplicate independent platings from a NUNC 6-well plate. For focus-forming assays, EEF1A2 and RAS^{val12}, both under the control of the CMV promoter, were transfected into Rat1 fibroblasts using calcium phosphate according to the manufacturer's directions (Gibco). The pCDNA3 empty vector was used as a control. Transfected cells were grown in 2% FBS/DMBM at 37° C. for 14 days and the media changed every three days. Transfection efficiency was determined by counting colonies that arise in selective media (Zeocin for EEF1A2 and G418 for Ras). Foci were counted by washing plates in PBS, fixing in 10% acetic acid and staining with 0.4% crystal violet. Counts are the mean of triplicate experiments, each containing triplicate independent transfections. For soft agarose assays, 2×10⁴ NIH 3T3 cells were placed in 3 ml of 0.35% low gelling temperature agarose (Sigma) in 10% FBS/DMEM and overlaid on 5 ml 0.8% agarose/10% FBS/DMEM in a 60 mm dish. Cells were grown at 37° C. for 14 days to allow colony formation.

### Example 4

### Tumor Xenografts

NIH 3T3 or ES-2 cells (1×10⁶) were injected subcutaneously into the hind leg of nude mice and the animals were sacrificed 21 days post injection. Tumor volume (V) was estimated from the length (l) and width (w) of the tumor by the formula: V=(π/6)×((l+w)/2)³. Tumors were fixed in formalin overnight at 4° C. and paraffin embedded. Sections were de-waxed and stained with Haematoxylin and Eosin. Animal experiments were conducted through protocols approved by the Central Animal Facility at McMaster University.

1. A method for diagnosing cancer in a subject comprising measuring EEF1A2 gene amplification, EEF1A2 mRNA levels, or EEF1A2 protein levels or activity in a biological sample obtained from the subject and comparing the measured EEF1A2 gene amplification, EEF1A2 mRNA levels, or EEF1A2 protein levels or activity with levels of EEF1A2 gene amplification, EEF1A2 mRNA levels, or EEF1A2 protein levels or activity in a control wherein an increase in the measured EEF1A2 gene amplification, EEF1A2 mRNA levels, or EEF1A2 protein levels or activity as compared to the control is indicative of the subject having cancer.

2. The method of claim 1 wherein the cancer is ovarian cancer, breast cancer or colorectal cancer.

3. The method of claim 1 wherein the biological sample comprises a serum or plasma sample or a tumor tissue biopsy sample obtained from the subject.

4. A method for prognosticating survival and selecting an effective treatment regime for a patient suffering from cancer comprising measuring EEF1A2 gene amplification, EEF1A2 mRNA levels, or EEF1A2 protein levels or activity in a biological sample obtained from the subject.

5. The method of claim 4 wherein the patient is suffering from ovarian, breast or colorectal cancer.

6. The method of claim 4 wherein the biological sample comprises a serum or plasma sample or a tumor tissue biopsy sample obtained from the patient.

7. A kit for prognosticating and/or diagnosing cancer comprising means for measuring EEF1A2 gene amplification, EEF1A2 mRNA levels, or EEF1A2 protein levels or activity in a biological sample.

8. A method for inhibiting expression of EFF1A2 of a tumor cell, comprising contacting the cell with an antisense oligonucleotide which inhibits expression of EEF1A2 by the cell, wherein a protein encoded by said EEF1A2 has an amino acid sequence comprising SEQ ID NO:6, and wherein the antisense oligonucleotide specifically inhibits expression of said EEF1A2 and does not inhibit expression of EEF1A1.

9. The method of claim 8, wherein the antisense oligonucleotide interacts in the cell with an mRNA molecule which encodes said EEF1A2 protein such that expression of the protein in the cell is inhibited.

10. The method of claim 8, wherein the antisense oligonucleotide is a ribozyme.

11. The method of claim 8, wherein the tumor cell is an ovarian, breast or colorectal tumor cell.

12. The method of claim 8, further comprising exposing the tumor cell to a therapeutic drug such that growth of the tumor cell is inhibited.

13. The method of claim 12, wherein the therapeutic drug comprises an anthracycline, epipodophyllotoxin, vinca alkaloid, or metallocene, or is cyclophosphamide, methotrexate, fluorouracil, doxorubicin, epirubicin, paclitaxel, cisplatin, or staurosporine.

14. The method of claim 12, wherein the therapeutic drug comprises a taxane.

15. The method of claim 8, wherein the antisense oligonucleotide is included in a recombinant expression vector which permits expression of the antisense oligonucleotide in the cell.

16. The method of claim 8, wherein the antisense oligonucleotide is administered in a viral vector or liposome.

17. The method of claim 8, wherein the antisense oligonucleotide binds to a coding region of a nucleic acid molecule which encodes said EEF1A2 protein.

18. The method of claim 8, wherein the antisense nucleic oligonucleotide binds to a non-coding region of a nucleic acid molecule which encodes said EEF1A2 protein.

19. The method of claim 8, wherein the antisense oligonucleotide is complementary in sequence to a regulatory region of a gene which encodes said EEF1A2 protein.

20. The method of claim 8, wherein the antisense oligonucleotide is complementary in sequence to a transcription initiation region of a gene which encodes said EEF1A2 protein.

21. The method of claim 8, wherein the antisense oligonucleotide is complementary in sequence to a region which precedes or spans the translation initiation codon of a gene which encodes said EEF1A2 protein.

22. The method of claim 8, wherein the antisense oligonucleotide is complementary in sequence to an untranslated region of a mRNA which encodes said EEF1A2 protein.

23. The method of claim 22, wherein the antisense oligonucleotide is complementary in sequence to a 3′ untranslated region of the mRNA.

24. A method for treating cancer comprising administering to a patient suffering from cancer an inhibitor of EEF1A2 expression and/or EEF1A2 activity.

25. The method of claim 24, wherein the inhibitor of EEF1A2 expression or EEF1A2 activity comprises an antisense oligonucleotide which inhibits expression of an EEF1A2 protein by a tumor cell.

26. The method of claim 24 or 25, further comprising administering a therapeutic drug.

27. The method of claim 26, wherein the therapeutic drug comprises an anthracycline, epipodophyllotoxin, vinca alkaloid, or metallocene, or is cyclophosphamide, methotrexate, fluorouracil, doxorubicin, epirubicin, paclitaxel, cisplatin, or staurosporine.

28. The method of claim 26, wherein the therapeutic drug comprises a taxane.

29. A screening assay to identify new anticancer agents comprising measuring an agent's ability to inhibit EEF1A2 expression and/or EEF1A2 activity.

30. The screening assay of claim 29 wherein the agent's ability to inhibit EEF1A2 expression and/or EEF1A2 activity is measured by EEF1A2-mediated enhancement of NIH 3T3 cell growth, EEF1A2-mediated enhancement of protein translation or EEF1A2-mediated microtubule cleavage.


SEQUENCE LISTING (9 sequences)

SEQ ID NO: 1 - protein, 14 aa, Artificial sequence
  Synthetic
GKPIPNPLLGLDST

SEQ ID NO: 2 - DNA, 20 bp, Artificial sequence
  Synthetic
cttttgctgggagtgtgagg

SEQ ID NO: 3 - DNA, 22 bp, Artificial sequence
  Synthetic
gctgggagtgtgtgaggggctg

SEQ ID NO: 4 - DNA, 19 bp, Artificial sequence
  Synthetic
ggttgctgtgggcttgagt

SEQ ID NO: 5 - DNA, 1841 bp, Homo sapien
actgcgccgccaccgtcaataggtggaccccctcccggagataaaaccgccggcgccggcgccgccagtccctctggctgagacctcggctccggaatcactgcagcccccctcgccctgagccagagcaccccgggtcccgccagcccctcacactcccagcaaaatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgaagcgcgggcgcccgcggcgcgaccctccccggcggcgccgcgctccgaaccccggcccggcccccgccccgcccccgccccgcgcgccgctccggcgccccgcacccccgccaggcgcatgtctgcacctccgcttgccagaggccctcggtcagcgactggatgctcgccatcaaggtccagtggaagttcttcaagaggaaaggcgcccccgccccaggcttccgcgcccagcgctcgccacgctcagtgcccgttttaccaataaactgagcgaccccag

SEQ ID NO: 6 - protein, 463 aa, Homo sapien
MGKEKTHINIVVIGHVDSGKSTTTGHLIYKCGGIDKRTIEKFEKEAAEMGKGSFKYAWVLDKLKAERERGITIDISLWKFETTKYYITIIDAPGHRDFIKNMITGTSQADCAVLIVAAGVGEFEAGISKNGQTREHALLAYTLGVKQLIVGVNKMDSTEPAYSEKRYDEIVKEVSAYIKKIGYNPATVPFVPISGWHGDNMLEPSPNMPWFKGWKVERKEGNASGVSLLEALDTILPPTRPTDKPLRLPLQDVYKIGGIGTVPVGRVETGILRPGMVVTFAPVNITTEVKSVEMHHEALSEALPGDNVGFNVKNVSVKDIRRGNVCGDSKSDPPQEAAQFTSQVIILNHPGQISAGYSPVIDCHTAHIACKFAELKEKIDRRSGKKLEDNPKSLKSGDAAIVEMVPGKPMCVESFSQYPPLGRFAVRDMRQTVAVGVIKNVEKKSGGAGKVTKSAQKAQKAGK

SEQ ID NO: 7 - DNA, 1837 bp, Homo sapien
tttttcgcaacgggtttgccgccagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - protein, 13 aa, Artificial sequence
  Synthetic
SHTTLLEAVDCIL

SEQ ID NO: 9 - protein, 13 aa, Artificial sequence
  Synthetic
SGVSLLEALDTIL